(19) United States
(12) Patent Application Publication
(10) Pub. No.: US 2008/0177092 A1
(43) Pub. Date: Jul. 24, 2008

(54) Continuous Method For The Production Of A Dioxirane

(75) Inventors: Mathieu Chabanas, Berg (DE); Joaquim Henrique Teles, Otterstadt (DE); Gunnar Heydrich, Limburgerhof (DE); William Ronald Sanderson, Warrigton Was (GB)

Correspondence Address: CONNOLLY BOVE LODGE & HUTZ, LLP, P O BOX 2207, WILMINGTON, DE, 19899, US

(73) Assignee: Basf Aktiengesellschaft, Ludwigshafen (DE)

(21) Appl. No.: 11/817,427

(22) PCT Filed: Mar. 1, 2006
(86) PCT No.: PCT/EP06/60352

§ 371 Date: Aug. 30, 2007

(30) Foreign Application Priority Data
Mar. 2, 2005 (DE) 10 2005 009 468.6

(51) Int. Cl. C07D 493/10

(52) U.S. Cl. 549/332

(57) ABSTRACT

Continuous processes comprising: oxidizing a ketone with a solution comprising active oxygen in a reactor in the presence of a buffer substance and a stripping gas to form a gas stream comprising a dioxirane and a liquid stream; and continuously drawing the gas stream comprising the dioxirane and the liquid stream from the reactor; wherein a liquid phase residence time of 1 minute to 4 hours and a normalized condensate mass flow rate of at least 500 g/mol of active oxygen are maintained.

The invention relates to a continuous process for preparing a dioxirane.

Dioxiranes, the simplest cyclic peroxides, are often used as neutral, highly reactive and highly selective oxidizing agents in organic chemistry. They have a series of advantages over other oxidizing agents, for example peracids: short reaction times, high yields, tolerance toward hydrolysis-sensitive groups or simplified workup.

Dioxiranes as oxidizing agents may either be generated in situ or be used as a solution in the starting ketone of the oxidation to the dioxirane, the latter variant having the broadest application spectrum. Frequently, solutions with a maximum of from 1 to 1.5% by weight of dioxirane in the corresponding starting ketone, often acetone, are used.

Dioxiranes are prepared by oxidizing ketones with solutions comprising active oxygen, in particular hydrogenmonopersulfate-containing solutions, in the presence of a buffer substance. Dioxirane solutions in the starting ketone are obtained from the reaction mixture by distillation and/or by stripping with an inert gas, in particular helium, argon or nitrogen.

Publications by Murray (J. Org. Chem. 1985, 50, 2847), Eaton (J. Org. Chem. 1988, 53, 5353) or Adam (Chem. Ber. 1991, 124, 2377) describe obtaining dimethyldioxirane solutions under semibatch conditions. While the stripping gas is often metered in continuously, at least one of the three reaction components, hydrogenmonopersulfate, acetone and buffer substance, is initially charged batchwise. These processes have disadvantages including the following: low dioxirane yields of a maximum of 5% based on the hydrogenmonopersulfate (in the above-cited publication of Adam) and also complicated process steps: monohydrogenpersulfate is metered in as a solid; the reaction mixture is heterogeneous; the reactor has to be flushed between two reactions.

WO 93/08144 describes an improved process for preparing dioxiranes, in which the starting ketone is used in the gas phase with the stripping gas and the oxidizing agent is an aqueous NaHSO₅ solution (prepared by neutralization of Caro's acid with NaOH). Even though these measures lead to a simplification and cheapening of the overall process, the yields of dimethyldioxirane based on the hydrogenmonopersulfate are still unsatisfactory (about 5% based on H₂SO₅).

It was accordingly an object of the invention to provide an improved continuous process for preparing dioxiranes, which in particular has a substantially higher yield based on the solution comprising active oxygen and which is thus suitable for industrial scale use.

The achievement consists in a continuous process for preparing a dioxirane in liquid phase in a reactor by oxidizing a ketone with a solution comprising active oxygen in the presence of a buffer substance and stripping out the resulting dioxirane with a stripping gas, in which the ketone, the solution comprising active oxygen, the buffer substance and the stripping gas are fed continuously to the reactor, and a gas stream comprising the dioxirane and also a liquid stream are drawn off continuously from the reactor, which comprises operating the reactor with a residence time of the liquid phase between 1 min and 4 h and with a normalized condensate mass flow rate of at least 500 g/mol of active oxygen used.

It has been found that it is essential for the efficient performance of the process for preparing dioxiranes firstly to ensure a residence time of the liquid phase in the reactor between 1 min and 4 h, preferably between 20 min and 1 h, more preferably between 30 and 40 min, and at the same time to operate the reactor in such a way that the normalized condensate mass flow rate is at least 500 g/mol of active oxygen used.

The residence time of the liquid phase in the reactor denotes, in a known manner, the volume of the liquid in the reactor divided by the total volume flow rate of all liquid reactants.

The normalized condensate mass flow rate is defined by the equation below:

$$Normalized condensate mass flow rate ( g \text{/} mol ) = \frac{{\dot{M}}_{condensate ( g \text{/} h )}}{{\dot{F}}_{active oxygen used ( mol \text{/} h )}}$$

in which \dot{M}_{condensate(g/h)} denotes the mass flow rate in g/h of the condensate which can be obtained by condensing all condensable components out of the gas stream drawn off from the reactor, the condensable components being all components having a boiling point above −20° C. at standard pressure,

and \dot{F}_{active oxygen used (mol/h)} the quantitative flow rate in mol/h of the active oxygen used.

Active oxygen is understood to mean all peroxidic oxygen atoms which are detected by means of iodometric titration. For instance, 1 mol/h of potassium hydrogenmonopersulfate or 1 mol/h of sodium hydrogenmonopersulfate or 1 mol/h of hydrogen peroxide each means 1 mol/h of active oxygen.

Instead of a single reactor, it is possible to use an arrangement with a plurality of reactors. In this case too, the above definition of the normalized condensate mass flow rate applies, the condensate comprises the condensable fractions from all gas streams drawn off from reactors forming the plant, and the mass flow rate to be used for the active oxygen used being the sum of all reactant streams which comprise active oxygen and are introduced into the reactors forming the plant.

The normalized condensate mass flow rate can be controlled in particular via the process conditions of temperature, pressure, mass flow rate and composition of the feeds, and also mass flow rate of the stripping gas.

It has been found that increased yields are obtained when the normalized condensate mass flow rate is at least 500 g of condensate/mole of active oxygen, preferably between 1000 and 10000 g of condensate/mole of active oxygen, more preferably between 2000 and 4000 g of condensate/mole of active oxygen.

In the process according to the invention, reactants—ketone, solution comprising active oxygen and buffer substance—and the stripping gas are fed continuously as fluid streams.

In the present context, a ketone is understood to mean an aliphatic, aromatic, araliphatic, cyclic or acyclic compound or a mixture of aliphatic, aromatic, araliphatic, cyclic or acyclic compounds having at least one ketone function. This compound or mixture of compounds may also comprise other functional groups, for example halogens, in particular chlorine or fluorine, hydroxyl groups, etc.

The ketone is preferably a substance or a mixture of substances having a boiling point of below 100° C. at standard pressure, in particular selected from the following list:

Acetone, 1,1,1-trifluoroacetone, butanone, diethyl ketone, cyclopentanone and cyclohexanone.

The ketone is more preferably acetone or 1,1,1-trifluoroacetone, in particular acetone.

The ketone may be used as pure compound in organic or aqueous solution or else in the gas phase, as a mixture with the stripping gas.

The ketone functions both as a reactant and as a solvent for the product.

The solution comprising active oxygen is in particular an aqueous or an organic solution comprising a hydrogenmonopersulfate species or a precursor thereof.

The hydrogenmonopersulfate species is in particular one or more substances selected from the following list: lithium hydrogenmonopersulfate, sodium hydrogenmonopersulfate, potassium hydrogenmonopersulfate, rubidium hydrogenmonopersulfate, cesium hydrogenmonopersulfate, tetraalkylammonium hydrogenmonopersulfate, preferably sodium hydrogenmonopersulfate and potassium hydrogenmonopersulfate.

The precursors used for monohydrogenpersulfate species may be compounds which are formed by reaction of peroxides with sulfuric acid, in particular Caro's acid and salts thereof or peroxodisulfuric acid and salts thereof.

Preferred hydrogenmonopersulfate-containing solutions are, for example, aqueous triple salt solutions, triple salt referring to the salt [2 KHSO₅.KHSO₄.K₂SO₄], known under the brand names Oxone®, Caroate® or Curox®, whose pH can be adjusted by admixing with an acid or a base, or else aqueous, partly neutralized Caro's acid solutions, as described in WO 93/08144. The Caro's acid may either be prepared in situ or ex situ, for example from sulfuric acid and hydrogen peroxide, in an H₂SO₅ generator.

The concentration of the hydrogenmonopersulfate-containing solution is selected preferably at >10⁻⁵ mol of HSO₅⁻/l, more preferably >10⁻³ mol of HSO₅⁻/l, in particular >10⁻² mol of HSO₅⁻/l.

The buffer substance used is a basic compound, in particular a hydroxide, carbonate, hydrogencarbonate, phosphate or hydrogenphosphate of alkali metals or alkaline earth metals, or mixtures thereof, more preferably sodium hydroxide, sodium hydrogencarbonate or potassium hydrogencarbonate.

The buffer substance is added preferably in a concentration and a mass flow rate such that the ratio of base equivalent to active oxygen in the solution comprising active oxygen is between 0.25 and 5 mol/mol, preferably between 1 and 3 mol/mol. Composition and mass flow rate of the buffer substance solution may also be adjusted pH-dependently, in particular in such a way that the pH in the liquid phase in the reactor is between 4 and 12, preferably between 6 and 10, more preferably between 7 and 9.

In the process according to the invention, the reactants and the stripping gas are metered in continuously. Continuously also refers to the addition of reactants, mixtures of reactants and of the stripping gas in portions. Reactants, mixtures of reactants or stripping gas can be added in portions simultaneously or sequentially. Two of the three reactants, ketone, solution comprising active oxygen and buffer substance, or even all three reactants may be added premixed, for example in a static mixer.

For the continuous performance of the process, all reactants are advantageously fed in liquid form. The concentrations of the reactants are advantageously selected such that there is no deposition of solids in the reactor.

The process may be performed in all reactor types or combinations of reactor types which can continuously contact a liquid phase and a gas phase and ensure mass transfer between gas phase and liquid phase. Preferred reactors are bubble columns (vertical or horizontal), columns with structured packings or trays, in which case it is unimportant which phase is the continuous phase, and also thin-film or falling-film evaporators, and also continuous stirred tanks with gas inlet or loop reactors.

The operating temperature of the reactor or of the reactors may be selected between minus 50° C. and 100° C., preferably between minus 10 and 70° C., more preferably between 0 and 30° C., and the operating pressure between 0.1 mbar absolute and 50 bar absolute, preferably between 1 mbar absolute and 5 bar absolute, particularly preferably between 0.1 bar absolute and 1.5 bar absolute and more preferably between 0.3 bar absolute and 1.2 bar absolute.

The contact between liquid phase and gas phase in the reactor can be effected in cocurrent, countercurrent or crosscurrent.

The gas stream drawn off from the reactor may be recycled partly or fully into the reactor.

It is likewise possible to recycle the liquid drawn off from the reactor partly or fully into the reactor.

It is also possible to use any combination of reactors in a dioxirane production unit. In this case too, the normalized condensate mass flow rate from the entire production unit has to be at least 500 g/mol of active oxygen.

The gas phase comprising dioxirane drawn off from the reactor or the reactors can be further processed directly, for example conducted to another reactor.

In a preferred embodiment, however, the gas phase comprising dioxirane is partially condensed to obtain a solution comprising dioxirane and an offgas which is preferably recycled at least partly as stripping gas. In this case, the proportion of the recycled offgas is preferably selected such that the oxygen concentration in the offgas downstream of the reactor does not exceed 12% by volume, preferably 9% by volume.

In one embodiment, a defoamer which is inert under reaction conditions, preferably a relatively long-chain alkane, in particular n-dodecane, n-tridecane, n-tettadecane or a mixture thereof, single-chain alcohols or silicone oils, more preferably n-dodecane, is metered into the reactants together with the ketone.

In principle, all known defoamers (foam inhibitors) may be used. The amount of the defoamer used is preferably a maximum of 5% by weight of the ketone used, in particular a maximum of 1% by weight of the ketone used.

Suitable stripping gases are all substances or substance mixtures which are gaseous and inert under reaction conditions. Preference is given to components or mixtures of components of air, in particular nitrogen or nitrogen/oxygen mixtures.

In an advantageous embodiment of the process, the water content of the gas stream comprising dioxirane is reduced by distillation. In this case, a water content of the liquid stream obtained after partial condensation of the components from the gas stream having a boiling point above −20° C. of below 1% by weight, preferably below 0.5% by weight, more preferably below 0.01% by weight, based on the total weight of the liquid stream, is attained.

For this purpose, it is possible to introduce the gas stream directly into a corresponding distillation column, or else to feed it first to a partial condensation which is preferably carried out in two stages. In this case, a first condenser (main condenser) is advantageously operated with the aid of cold water at temperatures above 0° C., and a postcondenser with brine at temperatures between −10 and −30° C. Advantageously, the postcondenser can be attached directly to the main condenser, so that the cold condensate of the postcondenser flows to the main condenser and thus supports its action.

In the partial condensation of the gas stream which comprises dioxirane and is drawn off from the reactor, a solution of the dioxirane in the starting ketone is obtained and is fed to a distillation column for the purpose of water depletion, and uncondensable fractions which are discharged as offgas. From the distillation column, a top stream comprising the dioxirane product of value is drawn off, which, after partial condensation of the components having a boiling point above −20° C., gives rise to a liquid stream having a water content of <1% by weight, preferably <0.5% by weight, more preferably <0.01% by weight.

The distillation column is preferably designed in such a way that the stripping section has more than three, in particular from 3 to 10 theoretical plates, and the rectifying section more than 10, in particular from 10 to 25 theoretical plates.

In the process variant in which the gas stream which comprises dioxirane and is drawn off from the reactor is introduced directly into the distillation column for the purpose of water depletion, the top stream drawn off from the distillation column is partly condensed, preferably in two stages, in a main condenser operated above 0° C. with water and a postcondenser operated at from −10 to −30° C. with brine to obtain a solution of the dioxirane in the starting ketone, and the uncondensable fractions are discharged as offgas.

In this process variant, the distillation column is designed as a stripping column, preferably having at least 5 theoretical plates. Higher numbers of plates improve the separation. Thus, from 10 to 20 theoretical plates are preferable.

The working pressure in the column does not differ substantially from the pressure at which the reactor is operated. In order to minimize the loss of ketone, it is therefore necessary to operate the condenser at the top of the column at low temperatures, advantageously at temperatures below 0° C., preferably at temperatures between −10 and −30° C.

A particularly inexpensive operating mode can be achieved when condensation is effected with at least two condensers at different temperature levels. For example, the first condenser can be operated with cold water at temperatures above 0° C., and a downstream condenser with brine at temperatures down to −30° C. The postcondenser may be attached directly to the main condenser, so that the cold condensate from the postcondenser can flow to the first condenser and support its action.

Irrespective of the process variant, there are in principle no restrictions with regard to the usable separating internals in the distillation column: suitable for this purpose are both random packings and structured packings or trays.

In order to minimize the pressure drop in the distillation column, random packings are particularly suitable, but also structured sheet metal or fabric packings, preferably having a specific surface area of from 100 to 750 m²/m³, more preferably of from 250 to 500 m²/m³.

In a particularly advantageous process variant, not only is the gas stream comprising dioxirane from the reactor worked up by distillation, but the liquid effluent from the reactor is also fed to a stripping column which has the task of removing product of value still present therein and also a majority of the starting ketone, and of feeding them back to the reactor for preparing dioxirane. The stripping column may have a bottom evaporator which is operated in such a way that the starting ketone evaporates and functions as stripping gas, but it may also be operated with an inert gas as stripping gas. The stripping column should have at least 5, preferably from 5 to 15 theoretical plates.

From the stripping column, a stream which is free of dioxirane and substantially free of starting ketone is drawn off and is discharged into the wastewater. The vapor stream from the stripping column is preferably recycled uncondensed into the reactor for preparing the dioxirane.

In an advantageous embodiment, the distillation column for water depletion and the stripping column may be configured as a single apparatus, in which case the distillation and the stripping section are separated from one another by a liquid collecting tray. The liquid which collects on the liquid collecting tray is advantageously recycled into the reactor for preparing dioxirane.

The collecting tray has suitable devices, especially chimneys, via which the vapors can ascend from the stripping section into the distillation section.

In a further preferred embodiment of the process, the gas or liquid stream which comprises dioxirane and has preferably been depleted of water is fed to an oxidation reactor in which a substrate having at least one oxidizable functional group is oxidized, preferably continuously, by the dioxirane to reduce it to the starting ketone, and it is removed from the oxidized substrate, for example by distillation in a falling-film evaporator or a distillation column, and recycled into the reactor for preparing the dioxirane. In this case, there are no restrictions with regard to the reactor type or the combination of reactors for the oxidation reaction with dioxirane. Particularly suitable apparatus are continuous stirred tanks or tubular reactors when the dioxirane is used as a liquid solution after partial condensation. When the dioxirane-containing gas stream is used directly, without partial condensation, bubble columns are particularly suitable.

Oxidizable substrates may generally be compounds having at least one oxidizable functional group. Preference is given to substrates having carbon-carbon double bonds, such as alkenes, enolates, or acyl, alkyl or silyl enol ethers. Preference is further given to substrates having sulfur-, nitrogen- or phosphorus-containing functional groups, such as sulfides, sulfoxides, amines, amides, hydroxylamines, phosphines or phosphites.

The substrates mentioned may be used as a pure compound, as a mixture or as a solution in a suitable solvent.

The invention is illustrated in detail below with reference to a drawing and also by working examples.

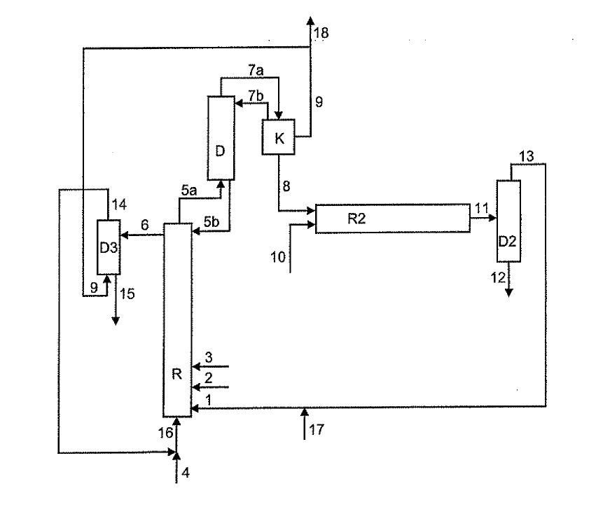

FIG. 1 shows the schematic illustration of a preferred plant for performing the process according to the invention.

A ketone or ketone/water mixture (stream 1), potassium hydrogenmonopersulfate solution (stream 2) and sodium hydrogencarbonate solution (stream 3), and also, via a frit, stripping gas (stream 4), are fed to a reactor R for preparing dioxirane. From the reactor, a gaseous top stream 5a comprising the dioxirane, stripping gas, ketone and water is drawn off and introduced into the lower region of a distillation column D. The bottom effluent, stream 5b, from the distillation column D is recycled into the reactor R. From the reactor R, a water-rich liquid stream 6 comprising water, salts, residues of ketone and traces of the dioxirane product of value is additionally drawn off. Stream 6 is introduced into a stripping column D3 to remove the residues of the ketone. From the stripping column D3, a ketone-containing vapor stream 14 comprising the stripping gas, ketone, traces of dioxirane and also traces of water is drawn off and, in the preferred working variant shown, recycled as stream 16 into the reactor R. From the stripping column D3, a bottom stream 15 comprising water and salts is drawn off and discharged into the wastewater.

From the distillation column D, a dewatered vapor stream 7a comprising dioxirane, stripping gas and ketone is drawn off, condensed in a condenser K, introduced partly as reflux 7b back to the distillation column D and otherwise introduced as dewatered dioxirane-containing liquid stream 8 comprising ketone and dioxirane into the oxidation reactor R2 for oxidizing a substrate 10, if appropriate in the presence of a solvent. The uncondensable gas stream 9 from the condenser K is partly discharged from the process (stream 18) and otherwise recycled into the stripping column D3 for removing the ketone residues. The effluent from the oxidation reactor R2, stream 11, is separated in an apparatus D2, which may be a distillation column or a falling-film evaporator, into a crude product 12 comprising the oxidized substrate and, if appropriate, solvents, and a top stream 13 comprising the ketone which is recycled into the reactor R for preparing the dioxirane.

### EXAMPLE 1

The following components were metered separately and continuously to the lower third of a bubble column which was operated at 20° C. and 0.3 bar absolute and had an internal diameter of 40 mm, a height of 630 mm and an empty pipe volume of approx. 0.79 l:
1) a 9.75% by weight aqueous potassium hydrogenmonopersulfate solution with a mass flow rate of 95.2 g/h, corresponding to 0.061 mol/h of hydrogenmonopersulfate, prepared from the triple salt [2 KHSO₅.KHSO₄.K₂SO₅] and water,
2) 158.7 g/h of a 7.06% by weight aqueous sodium hydrogencarbonate solution,
3) 414.2 g/h of a 57% by weight acetone solution in water comprising 400 ppm of n-dodecane as a defoamer and
4) via a frit, 180 l (STP)/h of nitrogen.

The potassium hydrogenmonopersulfate content of the aqueous solution was determined by iodometric titration shortly before use. From the bubble column, 478 g/h of a water-rich liquid and also a dioxirane-containing gas phase were withdrawn continuously. This gas phase was conducted through a very efficient cooler operated at −30° C. The cooler was dimensioned such that components having a boiling point above −20° C. were virtually fully condensable. After the condensation, 173.5 g/h of a solution comprising dioxirane, with 0.69% by weight of dimethyldioxirane and 8.3% by weight of water, and also a gas phase were obtained.

The dimethyldioxirane yield was 26.5% based on the hydrogenmonopersulfate.

The proportion of liquid in the bubble column was measured at the end of the experiment and was 60% by volume. This corresponds to a residence time of the liquid phase in the reactor of approx. 43 min.

The normalized condensate mass flow rate was approx. 2844 g of condensate/mole of active oxygen.

### EXAMPLE 2

Under the same conditions of temperature and pressure,
1) 90.0 g/h of a 5.74% by weight aqueous potassium hydrogenmonopersulfate solution, corresponding to 0.034 mol/h of hydrogenmonopersulfate, which was prepared in the same way as described under Example 1,
2) 155.7 g/h of a 3.53% by weight aqueous sodium hydrogencarbonate solution,
3) 413.8 g/h of a 57% by weight aqueous acetone solution comprising 400 ppm of dodecane as a defoamer and
4) via a frit, 180 l (STP)/h of nitrogen were fed to the same bubble column as described under Example 1.

From the bubble column, 472.7 g/h of a water-rich liquid and also a dioxirane-containing gas phase were withdrawn continuously. As described under Example 1, this gas phase was partially condensed to obtain 172.7 g/h of a solution comprising dimethyldioxirane with 0.46% by weight of dimethyldioxirane, and also a gas phase.

This corresponds to a dimethyldioxirane yield of 31.5% based on hydrogenmonopersulfate.

The proportion of liquid in the bubble column was measured at 62% by volume at the end of the experiment, corresponding to a residence time of the liquid phase in the reactor of approx. 45 min.

The normalized condensate mass flow rate in this example was approx. 5080 g of condensate/mole of active oxygen.

### EXAMPLE 3

The following components were metered continuously to the upper end of a bubble-cap tray column which was operated at 25° C. and 1 bar absolute and had 8 trays, a height of 800 mm and an internal diameter of 50 mm:
1) 23.9 g/h of a 9.59% by weight aqueous potassium hydrogemnonopersulfate solution, corresponding to 0.015 mol/h of hydrogenmonopersulfate, which was prepared in the same way as in Example 1,
2) 39.5 g/h of a 7.06% by weight aqueous sodium hydrogencarbonate solution, and
3) 85.6 g/h of a 57% by weight aqueous acetone solution.

At the same time, 200 l (STP)/h of nitrogen were metered in at the lower end of the bubble-cap tray column.

From the bubble-cap tray column, 96.8 g/h of a water-rich liquid were withdrawn continuously from the lower region thereof, and a gas phase comprising dioxirane from the upper region thereof. In a corresponding manner to Example 1, the gas phase was passed through a cooler to obtain 44.1 g/h of a solution comprising dimethyldioxirane, corresponding to 0.56% by weight of dimethyldioxirane, and also a gas phase. The dimethyldioxirane yield based on potassium hydrogenmonopersulfate was 22%.

In this example, the normalized condensate mass flow rate was approx. 2940 g of condensate/mole of active oxygen.

### EXAMPLE 4

A distillation column having an internal diameter of 30 mm, a height of 370 mm, charged with 5×5 mm glass Raschig rings was attached to the upper section of the bubble column described in Example 1. The reflux ratio was controlled via the temperature of a cooler at the top of the column (−10° C.). The reaction was operated under the same operating conditions as described under Example 1, i.e. at 20° C. and 0.3 bar absolute.

At the lower third of the bubble column, the following components were fed in continuously:
1) a 9.84% by weight aqueous potassium hydrogenmonopersulfate solution with a mass flow rate of 94.8 g/h, corresponding to 0.0613 mol/h of hydrogenmonopersulfate, prepared from the triple salt [2KHSO₅.KHSO₄.K₂SO₅] and water,
2) 157.3 g/h of a 7.06% by weight aqueous sodium hydrogencarbonate solution,
3) 414.9 g/h of a 57% by weight acetone solution in water comprising 400 ppm of dodecane, and
4) via a frit, 180 l (STP)/h of nitrogen.

From the bubble column, 152.0 g/h of a water-rich liquid and a dioxirane-containing gas phase were withdrawn continuously. The gas phase was conducted through the same cooler as described under Example 1. After partial condensation in the cooler, 173.5 g/h of a solution comprising dimethyldioxirane with 0.72% by weight of dimethyldioxirane (determined by iodometric titration) and 0.6% by weight of water (determined by standard Karl-Fischer titration), and also a gas phase comprising uncondensable constituent were obtained.

The dimethyldioxirane yield was 24% based on the hydrogenmonopersulfate used.

The liquid fraction in the bubble column was measured at the end of the experiment and was 64% by volume; this corresponds to a residence time of the liquid phase in the reactor of approx. 46 min.

The normalized condensate mass flow rate was approx. 2490 g of condensate/mole of active oxygen.

### COMPARATIVE EXAMPLE

In a semibatch process, a 4 l four-neck flask was initially charged with 635 g of water, 380 g of acetone and 145 g of sodium hydrogencarbonate.

The heterogeneous (liquid/solid) mixture was cooled to from 0 to 5° C.

At this temperature, the triple salt [2KHSO₅.KHSO₄.K₂SO₅], 300 g, corresponding to approx. 0.976 mol of hydrogenmonopersulfate, were added as a solid slowly and in portions via a solid metering unit.

The mixture was warmed to room temperature and a solution comprising dimethyldioxirane was subsequently distilled off with intensive stirring at 100 mbar and 25° C.

The dimethyldioxirane solution was isolated with the aid of a series of cold traps cooled to −78° C.

Within 80 min, after combination of the fractions obtained, 292.1 g of solution were isolated. The iodometric titration of this solution gave 0.93% by weight of dimethyldioxirane with, correspondingly, a total of 0.0367 mol of dimethyldioxirane. This corresponds to a dimethyldioxirane yield based on the hydrogenmonopersulfate used of approx. 3.8%. The comparative example was carried out analogously to the method of Adam in Chem. Ber. 1991, 124, 2377.

The mean normalized condensate mass flow rate was 299 g of condensate/mole of active oxygen.

1-24. (canceled)

25. A continuous process comprising:
oxidizing a ketone with a solution comprising active oxygen in a reactor in the presence of a buffer substance and a stripping gas to form a gas stream comprising a dioxirane and a liquid stream; and
continuously drawing the gas stream comprising the dioxirane and the liquid stream from the reactor;
wherein a liquid phase residence time of 1 minute to 4 hours and a normalized condensate mass flow rate of at least 500 g/mol of active oxygen are maintained.

26. The process according to claim 25, wherein the normalized condensate mass flow rate is 1000 to 10000 g/mol of active oxygen.

27. The process according to claim 25, wherein the ketone comprises a compound having at least one ketone functional group and the compound is selected from the group consisting of aliphatic compounds, aromatic compounds, araliphatic compounds, cyclic compounds, acyclic compounds and mixtures thereof.

28. The process according to claim 25, wherein the ketone has a boiling point below 100° C. at standard pressure.

29. The process according to claim 25, wherein the ketone comprises a compound selected from the group consisting of acetone, 1,1,1-trifluoroacetone, butanone, diethyl ketone, cyclopentanone, cyclohexanone and mixtures thereof.

30. The process according to claim 25, wherein the solution comprising active oxygen comprises a hydrogenmonopersulfate species or a precursor thereof.

31. The process according to claim 30, wherein the hydrogenmonopersulfate species comprises a substance selected from the group consisting of lithium hydrogenmonopersulfate, sodium hydrogenmonopersulfate, potassium hydrogenmonopersulfate, rubidium hydrogenmonopersulfate, cesium hydrogenmonopersulfate, tetraalkylammonium hydrogenmonopersulfate, and mixtures thereof.

32. The process according to claim 30, wherein the hydrogenmonopersulfate species comprises a substance selected from the group consisting of sodium hydrogenmonopersulfate, potassium hydrogenmonopersulfate and mixtures thereof.

33. The process according to claim 30, wherein the hydrogenmonopersulfate precursor comprises a reaction product of a peroxide and sulfuric acid.

34. The process according to claim 30, wherein the hydrogenmonopersulfate species or the precursor thereof has a concentration of >10⁻⁵ mol/L in the solution.

35. The process according to claim 25, wherein the buffer substance comprises a basic compound.

36. The process according to claim 35, wherein the buffer substance is added in a concentration and at a mass flow rate such that a ratio of base equivalents to active oxygen in the solution comprising active oxygen is 0.25 to 5.

37. The process according to claim 35, wherein the buffer substance is added in a concentration and at a mass flow rate such that a liquid phase pH in the reactor is 4 to 12.

38. The process according to claim 25, wherein the reactor is operated at a pressure of 0.1 mbar absolute to 5 bar absolute and a temperature of −50 to +100° C.

39. The process according to claim 25, wherein the stripping gas comprises one or more components of air.

40. The process according to claim 25, wherein the reactor is selected from the group consisting of a bubble column, a column with structured packings or trays, a thin-film evaporator, a falling-film evaporator, a stirred tank, and a loop reactor.

41. The process according to claim 25, wherein the gas stream drawn from the reactor is subjected to partial condensation such that components having a boiling point above −20° C. at standard pressure are condensed to form a solution comprising dioxirane and an offgas.

42. The process according to claim 41, wherein a portion of the stripping gas is recycled such that an oxygen content of <12% by volume remains in the offgas after the partial condensation.

43. The process according to claim 25, wherein the oxidation of the ketone with the solution comprising active oxygen is carried out in the presence of a defoamer.

44. The process according to claim 25, wherein the gas stream comprising dioxirane drawn from the reactor is introduced directly into a distillation column from which a top stream comprising the dioxirane product of value is drawn off and, after partial condensation of the components having a boiling point above minus 20° C., gives rise to a liquid stream which comprises less than 1% by weight of water based on the total weight of the liquid stream.

45. The process according to claim 25, wherein the liquid stream from the reactor is introduced into a stripping column, the stripping column having at least 6 theoretical plates, and a bottom stream which is substantially free of starting ketone is removed from the stripping column and is discharged into the wastewater, and a top stream is removed from the stripping column.

46. The process according to claim 45, wherein the top stream comprising dioxirane is partly condensed in two stages to obtain a liquid solution of the dioxirane in the starting ketone, and an uncondensable fraction of the top stream is discharged partly as offgas and otherwise recycled as a stream into the stripping column.

47. The process according to claim 25, wherein the gas stream comprising dioxirane drawn from the reactor is first partly condensed in two stages to obtain a liquid solution of the dioxirane in the starting ketone which is fed to a distillation column from which a top stream comprising the dioxirane product of value is drawn off, and partial condensation of the components from the top stream having a boiling point above minus 20° C. forms a liquid stream having a water content of <1% by weight based on the total weight of the liquid stream.

48. The process according to claim 47, wherein the distillation column has a stripping section having at least 3 theoretical plates, and a rectifying section having at least 10 theoretical plates.

49. The process according to claim 25, further comprising feeding the gas stream comprising dioxirane to a subsequent oxidation reactor in which a substrate having at least one oxidizable functional group is oxidized by the dioxirane to reduce the dioxirane to the ketone which is removed from the oxidized substrate and recycled into the reactor.
